(19) United States
(12) Patent Application Publication
(10) Pub. No.: US 2003/0229145 A1
(43) Pub. Date: Dec. 11, 2003

(54) Pain treatment methods and compositions

(75) Inventors: Boris Chizh, Aachen (DE); Katharina Claudia Puetz, Duren (DE); Michael Haurand, Aachen (DE)

Correspondence Address: CROWELL & MORING LLP, INTELLECTUAL PROPERTY GROUP, P.O. BOX 14300, WASHINGTON, DC, 20044-4300, US

(73) Assignee: GRUENENTHAL GMBH, Aachen (DE)

(21) Appl. No.: 10/402,129

(22) Filed: Mar. 31, 2003

Related U.S. Application Data
(63) Continuation of application No. PCT/EP01/11230, filed on Sep. 28, 2001.

(30) Foreign Application Priority Data
Sep. 30, 2000 (DE) 100 48 715.7

(51) Int. Cl. A61K 31/198; C07C 229/08; C07C 229/28

(52) U.S. Cl. 514/561; 562/507; 562/553; 554/103

(57) ABSTRACT

The invention relates to amino acids, a method for the production thereof, medicaments containing said amino acids and the use of amino acids in the production of medicaments for treating pain.

### CROSS REFERENCE TO RELATED APPLICATIONS

The present application is a continuation of International Patent Application No. PCT/EP01/11230, filed Sep. 28, 2001, designating the United States of America and published in German as WO 02/30871 A1, the entire disclosure of which is incorporated herein by reference. Priority is claimed based on Federal Republic of Germany Patent Application No. 100 48 715.7, filed Sep. 30, 2000.

### FIELD OF THE INVENTION

The present invention relates to amino acids, methods for their manufacture, medicaments containing these compounds and the use of amino acids to manufacture medicaments for treating pain.

### BACKGROUND AND SUMMARY OF THE INVENTION

The cyclic GABA analogue gabapentin is a clinically proven antiepileptic. Gabapentin (GBP) also has further beneficial, medically relevant properties, especially as an analgesic. Novel structure classes which have affinity with the gabapentin binding sites are therefore of interest. With the above indications, there is a further need for substances which show correspondences with gabapentin in terms of their properties, for example in terms of their analgesic effect.

The treatment of chronic and non-chronic pain conditions is very important in medicine. There is a worldwide need for highly effective pain therapies. The pressing requirement for patient-compatible and target-oriented treatment of chronic and non-chronic pain conditions, which is to be understood as meaning successful and satisfactory pain treatment for the patient, is documented in a large number of scientific works which have recently appeared in the field of applied analgesia or fundamental research into nociception.

Classic opioids such as morphine are highly effective in the therapy of strong to very strong pains. Their use, however, is limited by the known side-effects, for example respiratory depression, vomiting, sedation, obstipation and tolerance development. Furthermore, they are less effective for neuropathic or incidental pains, from which especially tumor patients suffer.

### DESCRIPTION OF THE INVENTION

It was therefore an object of the invention to find structures, preferably novel structures, which have affinity with the gabapentin binding site and/or corresponding physiological efficacies, for example in respect of analgesia, or also other GBP indications.

The invention therefore provides the use of an amino acid of Formula I,

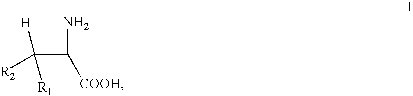

in which

R¹ and R² are each selected, independently of one another, from H; branched or unbranched, saturated or unsaturated, unsubstituted or mono- or polysubstituted C₁₋₁₀ alkyl; aryl, C₁₋₁₀ cycloalkyl or heteroaryl, in each case unsubstituted or mono- or polysubstituted; or

R¹ and R² together form a saturated or unsaturated, substituted or unsubstituted (CH₂)₃₋₆ ring, in which 0-2 C atoms may be replaced by S, O or NR⁴,

with R⁴ being: H; or saturated or unsaturated, branched or unbranched, mono- or polysubstituted or unsubstituted C₁-₁₀ alkyl;

optionally in the form of its racemates, its pure stereoisomers, especially enantiomers or diastereomers, or in the form of mixtures of the stereoisomers, especially the enantiomers or diastereomers, in any mixing ratio; in the form given or in the form of its acids or its bases or in the form of its salts, especially the physiologically acceptable salts, or in the form of its solvates, especially the hydrates;

to manufacture a medicament for treating pain, especially neuropathic, chronic or acute pain, epilepsy and/or migraines

or

to manufacture a medicament for treating hyperalgesia and allodynia, especially thermal hyperalgesia, mechanical hyperalgesia and allodynia and cold allodynia, or inflammatory or postoperative pain

or

to manufacture a medicament for treating hot flashes, postmenopausal complaints, amyotrophic lateral sclerosis (ALS), reflex sympathetic dystrophy (RSD), spastic paralysis, restless leg syndrome, acquired nystagmus; psychiatric or neuropathological disorders, such as bipolar disorders, anxiety, panic attacks, mood fluctuations, manic behavior, depression, manic-depressive behavior; painful diabetic neuropathy, symptoms and pain due to multiple sclerosis or Parkinson's disease, neurodegenerative diseases, such as Alzheimer's disease, Huntington's disease, Parkinson's disease and epilepsy; gastric intestinal injury; erythromelalgic or post-poliomyelitic pain, trigeminal or post-therapeutic neuralgia; or as an anticonvulsive, analgesic or anxiolytic.

In one embodiment of the invention, in the amino acids according to Formula I,

R¹ and R² are, independently of one another, H; or branched or unbranched, saturated or unsaturated, unsubstituted or mono- or polysubstituted C₁₋₁₀ alkyl; preferably, one of the residues R¹ and R² denotes C₁₋₂ alkyl and the other denotes C₂₋₁₀ alkyl, preferably unsubstituted, unbranched and saturated, or

R¹ and R² together form cyclopropyl, cyclopentyl, cyclohexyl or cycloheptyl.

In a particularly preferred embodiment of the invention, in the amino acids according to Formula I,

R¹ and R² are, independently of one another, branched or unbranched, saturated or unsaturated, unsubstituted or mono- or polysubstituted C₁₋₁₀ alkyl; aryl, C₃₋₁₀ cycloalkyl or heteroaryl, in each case, unsubstituted or mono- or polysubstituted;

or

R¹ and R² together form a ring and denote substituted or unsubstituted (CH₂)₃₋₆, in which 0-2 C atoms may be replaced by S, O or NR⁴,

with R⁴ being: H; or saturated or unsaturated, branched or unbranched, mono- or polysubstituted or unsubstituted C₁₋₁₀ alkyl.

These substances bind to the gabapentin binding site and exhibit a pronounced analgesic effect.

In the context of this invention, the terms alkyl or cycloalkyl residues mean saturated and unsaturated (but not aromatic), branched, unbranched and cyclic hydrocarbons, which may be unsubstituted or mono or polysubstituted. In this case, C₁₋₂ alkyl stands for C₁ or C₂ alkyl, C₁₋₃ alkyl stands for C₁, C₂ or C₃ alkyl, C₁₋₄ alkyl stands for C₁, C₂, C₃ or C₄ alkyl, C₁₋₅ alkyl stands for C₁, C₂, C₃, C₄ or C₅ alkyl, C₁₋₆ alkyl stands for C₁, C₂, C₃, C₄, C₅ or C₆ alkyl, C₁₋₇ alkyl stands for C₁, C₂, C₃, C₄, C₅, C₆ or C₇ alkyl, C₁₋₈ alkyl stands for C₁, C₂, C₃, C₄, C₅, C₆, C₇ or C₈ alkyl, C₁₋₁₀ alkyl stands for C₁, C₂, C₃, C₄, C₅, C₆, C₇, C₈, C₉ or C₁₀ alkyl and C₁₋₈ alkyl stands for C₁, C₂, C₃, C₄, C₅, C₆, C₇, C₈, C₉, C₁₀, C₁₁, C₁₂, C₁₃, C₁₄, C₁₅, C₁₆, C₁₇ or C₁₈ alkyl. Furthermore, C₃₋₄ cycloalkyl stands for C₃ or C₄ cycloalkyl, C₃₋₅ cycloalkyl stands for C₃, C₄ or C₅ cycloalkyl C₃₋₆ cycloalkyl stands for C₃, C₄, C₅ or C₆ cycloalkyl, C₃-₇ cycloalkyl stands for C₃, C₄, C₅, C₆ or C₇ cycloalkyl, C₃₋₈ cycloalkyl stands for C₃, C₄, C₅, C₆, C₇ or C₈ cycloalkyl, C₄₋₅ cycloalkyl stands for C₄ or C₅ cycloalkyl, C₄₋₆ cycloalkyl stands for C₄, C₅ or C₆ cycloalkyl, C₄₋₇ cycloalkyl stands for C₄, C₅, C₆ or C₇ cycloalkyl, C₅₋₆ cycloalkyl stands for C₅ or C₆ cycloalkyl and C₅₋₇ cycloalkyl stands for C₅, C₆ or C₇ cycloalkyl. With reference to cycloalkyl, the term also includes saturated cycloalkyls in which one or 2 carbon atoms are replaced by a heteroatom, S, N or O. The term cycloalkyl moreover especially covers mono- or poly-, preferably mono-, unsaturated cycloalkyls without any heteroatom in the ring, so long as the cycloalkyl does not constitute an aromatic system. The alkyl or cycloalkyl residues are preferably methyl, ethyl, vinyl (ethenyl), propyl, allyl (2-propenyl), 1-propinyl, methylethyl, butyl, 1-methylpropyl, 2-methylpropyl, 1,1 -dimethylethyl, pentyl, 1,1 -dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, or alternatively adamantyl, CHF₂, CF₃ or CH₂OH as well as pyrazolinone, oxopyrazolinone, [1,4]dioxane or dioxolane.

In this case, in connection with alkyl and cycloalkyl—unless otherwise expressly defined—the term substituted in the context of this invention means the substitution of at least one (and optionally several) hydrogen residues by F, Cl, Br, I, NH₂, SH or OH, the term “polysubstituted”, or “substituted” in the case of multiple substitution, being intended to mean that the substitution takes place multiply, both on different and on the same atoms, with the same or different substituents, for example triply on the same C atom as in the case of CF₃, or at different sites as in the case of —CH(OH)—CH═CH—CHCl₂. F, Cl and OH are especially preferred substituents here. With reference to cycloalkyl, the hydrogen residue may also be replaced by OC₁₋₃ alkyl or C₁₋₃ alkyl (in each case mono- or polysubstituted or unsubstituted), especially methyl, ethyl, n-propyl, i-propyl, CF₃, methoxy or ethoxy.

The term (CH₂)₃₋₆ is intended to mean —CH₂—CH₂—CH₂—, —CH₂—CH₂—CH₂—, —CH₂—CH₂—CH₂—CH₂—CH₂— and —CH₂—CH₂—CH₂—CH₂—CH₂—CH₂—, (CH₂)₁₋₄ is intended to mean —CH₂—, —CH₂—CH₂—, —CH₂—CH₂—CH₂— and —CH₂—CH₂—CH₂—CH₂—, (CH₂)₄₋₅ is intended to mean —CH₂—CH₂—CH₂—CH₂— and —CH₂—CH₂—CH₂—CH₂—CH₂—, etc.

The term aryl residue means ring systems with at least one aromatic ring but without heteroatoms in any of the rings at all. Examples are phenyl, naphthyl, fluoranthenyl, fluorenyl, tetralinyl or indanyl, especially 9H-fluorenyl or anthracenyl residues, which may be unsubstituted or mono- or polysubstituted.

The term heteroaryl residue means heterocyclic ring systems which have at least one unsaturated ring and contain one or more heteroatoms from the group nitrogen, oxygen and/or sulfur, and which may also be mono- or polysubstituted. Examples from the group of heteroaryls are furan, benzofuran, thiophene, benzothiophene, pyrrole, pyridine, pyrimidine, pyrazine, quinoline, isoquinoline, phthalazine, benzo[1,2,5]thiadiazole, benzothiazole, indole, benzotriazole, benzodioxolane, benzodioxane, carbazole, indole and quinazoline.

In this case, in connection with aryl and heteroaryl, the term substituted means substitution of the aryl or heteroaryl with R²³, OR²³, a halogen, preferably F and/or Cl, a CF₃, a CN, an NO₂, an NR²⁴R²⁵, a C₁₋₆ alkyl (saturated), a C₁₋₆ alkoxy, a C₃₋₈ cycloalkoxy, a C₃₋₈ cycloalkyl or a C₂₋₆ alkylene.

In this case, the residue R²³ stands for H, a C₁₋₁₀ alkyl, preferably a C₁₋₆ alkyl, an aryl or heteroaryl, or for an aryl or heteroaryl residue joined via saturated or unsaturated C₁₋₃ alkyl or via a C₁₋₃ alkylene group, in which case these aryl and heteroaryl residues may not themselves be substituted with aryl or heteroaryl residues,

the residues R²⁴ and R²⁵, which are identical or different, stand for H, a C₁₋₁₀ alkyl, preferably a C₁₋₆ alkyl, an aryl, a heteroaryl, or an aryl or heteroaryl residue joined via saturated or unsaturated C₁₋₃ alkyl or via a C₁₋₃ alkylene group, in which case these aryl and heteroaryl residues may not themselves be substituted with aryl or heteroaryl residues,

or the residues R²⁴ and R²⁵ together denote CH₂CH₂OCH₂CH₂, CH₂CH₂NR²⁶CH₂CH₂ or (CH₂)₃₋₆, and

the residue R²⁶ for H, a C₁₋₁₀ alkyl, preferably a C₁₋₆ alkyl, an aryl or a heteroaryl residue, or for an aryl or heteroaryl residue joined via saturated or unsaturated C₁₋₃ alkyl or via a C₁₋₃ alkylene group, in which case these aryl and heteroaryl residues may not themselves be substituted with aryl or heteroaryl residues.

The term salt is intended to mean any form of the active agent according to the invention in which it adopts an ionic form or is charged, and is coupled to a counterion (a cation or anion) or is in solution. This is also intended to include complexes of the active agent with other molecules and ions, especially complexes which are complexed via ionic interactions. This especially includes physiologically acceptable salts with cations or bases and physiologically acceptable salts with anions or acids.

The term physiologically acceptable salts with cations or bases means, in the context of this invention, salts of at least one of the compounds according to the invention—usually a (deprotonated) acid—as the anion with at least one, preferably inorganic, cation, which are physiologically acceptable—especially when used in humans and/or mammals. Particularly preferred are the salts of the alkali metals and alkaline-earth metals, or alternatively with NH₄⁺, but especially (mono-) or (di-) sodium, (mono-) or (di-) potassium, magnesium or calcium salts.

The term physiologically acceptable salts with anions or acids means, in the context of this invention, salts of at least one of the compounds according to the invention—usually protonated, for example with nitrogen—as the cation with at least one anion, which are physiologically acceptable—especially when used in humans and/or mammals. In the context of this invention, this especially includes the salt formed with a physiologically acceptable acid, that is to say salts of the respective active agents with inorganic or organic acids, which are physiologically acceptable—especially when used in humans and/or mammals. Examples of physiologically acceptable salts of particular acids are salts of: hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, 1,1-dioxo-1,2-dihydrolλ⁶-benzo[d]isothiazol-3-one (saccharinic acid), monomethylsebacic acid, 5-oxo-proline, hexane-1-sulfonic acid, nicotinic acid, 2-, 3- or 4-aminobenzoic acid, 2,4,6-trimethyl-benzoic acid, α-lipoic acid, acetylglycine, acetylsalicylic acid, hippuric acid and/or aspartic acid. The hydrochloride salt is particularly preferred.

All substances mentioned above and defined for use displace gabapentin from its binding site of a corresponding receptor - which is actually not yet known scientifically. This implies, however, that the substances according to the invention bind to the same binding site and will act physiologically via such binding, presumably with the same activity profile as gabapentin. The fact that this assumption of the same activity at the same binding site is actually correct is substantiated by the analgesic effect. The compounds according to the invention hence not only displace gabapentin from its binding site but also have a significant analgesic effect—like gabapentin. The invention therefore provides the use of the above defined amino acids in the aforementioned indications in which gabapentin is effective, especially in pain therapy, for epilepsy or migraines, but also particularly in neuropathic pain, i.e., hyperalgesia and allodynia, and the other gabapentin indications.

Gabapentin is a known antiepileptic with anticonvulsive activity. Besides this, gabapentin is also used in various other indications, inter alia being prescribed by treating doctors for migraines and bipolar disorders as well as hot flashes (for example in postmenopause) (M. Schrope, Modern Drug Discovery, September 2000, p. 11). Other indications in which gabapentin exhibits a therapeutic potential have been identified during human studies and in clinical use (J. S. Bryans, D. J. Wustrow; “3-Substituted GABA Analogs with Central Nervous System Activity: A Review” in Med. Res. Rev. (1999), pp. 149-177). This review article lists the activity of gabapentin in detail. For instance, gabapentin is effective in the treatment of chronic pains and behavioral disorders. The following may especially be mentioned: anticonvulsive and antiepileptic effects, use against chronic, neuropathic pain, especially thermal hyperalgesia, mechanical allodynia, cold allodynia. It furthermore acts against neuropathy triggered by nerve damage, and especially neuropathic pain, as well as inflammatory and postoperative pain. Gabapentin is also successful for antipsychotic effects, especially as an anxiolytic. Further verified indications comprise: amyotrophic lateral sclerosis (ALS), reflex sympathetic dystrophy (RSD), spastic paralysis, restless leg syndrome, treatment of symptoms and pain due to multiple sclerosis, acquired nystagmus, treatment of the symptoms of Parkinson's disease, painful diabetic neuropathy and psychiatric disorders, for example bipolar disorders, mood fluctuations, manic behavior. The use of gabapentin has furthermore been successful for erythromelalgic pain, post-poliomyelitic pain, trigeminal neuralgia and post-therapeutic neuralgia; (Bryans and Wustrow (1999), supra). The general efficacy in neurodegenerative diseases is also generally known and to be found with reference to the examples in Bryans and Wustrow (1999). Such neurodegenerative diseases include Alzheimer's disease, Huntington's disease, Parkinson's disease and epilepsy. The efficacy of gabapentin for gastrointestinal injury is also known.

In a preferred embodiment of the invention, amino acids according to Formula I are used, for which

R¹ and R² are, independently of one another, branched or unbranched, saturated or unsaturated, unsubstituted or mono- or polysubstituted C₁₋₁₀ alkyl; aryl, C₄₋₈ cycloalkyl or heteroaryl, in each case unsubstituted or mono- or polysubstituted;

or

R¹ and R² together form a ring and denote substituted or unsubstituted (CH₂)₅, so that a substituted or unsubstituted cyclohexyl is obtained.

In a particularly preferred embodiment of the invention, amino acids according to Formula I are used, for which

one of R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl; or aryl/heteroaryl, especially phenyl, naphthyl, furanyl, thiophenyl, pyrimidinyl or pyridinyl, in each case unsubstituted or monosubstituted (preferably with OCH₃, CH₃, OH, SH, CF₃, F, Cl, Br or I); or C₃₋₈ cycloalkyl, especially cyclopropyl, cyclobutyl, cyclopentyl, cyclohexyl or cycloheptyl, in each case unsubstituted or monosubstituted,

preferably

one of the residues R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl; or C₄₋₇ cycloalkyl, preferably cyclobutyl, cyclopentyl, cyclohexyl or cycloheptyl, especially cyclobutyl, cyclopentyl or cyclohexyl, in each case unsubstituted or monosubstituted,

especially

one of the residues R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl.

In a likewise particularly preferred embodiment of the invention, amino acids according to Formula I are (also) used, for which

R¹ and R² together form a ring and denote substituted or unsubstituted (CH₂)₅, so that a substituted or unsubstituted cyclohexyl is obtained,

preferably

R¹ and R² together form a ring and denote substituted or unsubstituted (CH₂)₅, so that a monosubstituted or unsubstituted cyclohexyl is obtained, especially an unsubstituted or methyl-substituted cyclohexyl.

The methods of the present invention exclude the use of some of the aforementioned compounds. Hence, in a preferred embodiment according to the invention, a compound or compound group according to Formula I are excluded under a least one of the following conditions:

R¹ and R² are both CH₃,

one of R¹ and R² is CH₃ and the other is C₂H₅,

one of R¹ and R² is CH₃ and the other is substituted phenyl,

R¹ and R² together form a ring and denote (CH₂)₄, hence forming a substituted or unsubstituted cyclopentyl ring, and

R¹ and R² together form a ring and denote (CH₂)₅, hence forming a substituted or unsubstituted cyclohexyl ring.

In a preferred embodiment of the invention, methods according to the present invention use amino acids that are selected from the following group:

2-amino-3-methylheptanoic acid

2-amino-3-methyloctanoic acid

2-amino-3-methylnonanoic acid

2-amino-3-methyldecanoic acid

2-amino-3-ethylhexanoic acid

2-amino-3-methylundecanoic acid

2-amino-3-cyclobutyl-butanoic acid

2-amino-3-cyclohexyl-butanoic acid

amino-(3-methyl-cyclohexyl)ethanoic acid, and

amino-(2-methyl-cyclohexyl)ethanoic acid.

These amino acid may be in the form of their racemates, their pure stereoisomers, especially enantiomers or diastereomers, or in the form of mixtures of the stereoisomers, especially the enantiomers or diastereomers, in any mixing ratio; in the form of their acids or their bases or in the form of their salts, especially the physiologically acceptable salts, preferably the hydrochloride or the sodium salt; or in the form of their solvates, especially the hydrates.

The invention also provides amino acids of Formula I,

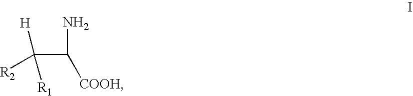

in which

R¹ and R² are, independently of one another, H; branched or unbranched, saturated or unsaturated, unsubstituted or mono- or polysubstituted C₁₋₁₀ alkyl; aryl, C₁₋₁₀ cycloalkyl or heteroaryl, in each case unsubstituted or mono- or polysubstituted; or

R¹ and R² together form a saturated or unsaturated, substituted or unsubstituted (CH₂)₃₋₆ ring, in which 0-2 C atoms may be replaced by S, O or NR⁴,

with R⁴ being: H; or saturated or unsaturated, branched or unbranched, mono- or polysubstituted or unsubstituted C₁₋₁₀ alkyl.

These amino acids may be in the form of their racemates, their pure stereoisomers, especially enantiomers or diastereomers, or in the form of mixtures of the stereoisomers, especially the enantiomers or diastereomers, in any mixing ratio; in the form of their acids or their bases or in the form of their salts, especially the physiologically acceptable salts, or in the form of their solvates, especially the hydrates.

In one embodiment, in the amino acids according to Formula I according to the invention,

R¹ and R² are each selected, independently of one another, H; or branched or unbranched, saturated or unsaturated, unsubstituted or mono- or polysubstituted C₁₋₁₀ alkyl; preferably, one of R¹ and R² denotes C₁₋₂ alkyl and the other denotes C₂₋₁₀ alkyl, preferably unsubstituted, unbranched and saturated, or

R¹ and R² together form cyclopropyl, cyclopentyl, cyclohexyl or cycloheptyl.

In a preferred embodiment, in the amino acids according to Formula I,

one of R¹ and R² denotes C₁₋₃ alkyl, especially methyl, ethyl, n-propyl or i-propyl, in each case unsubstituted or mono- or polysubstituted; and the other of R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl; or arylaheteroaryl, especially phenyl, naphthyl, furanyl, thiophenyl, pyrimidinyl or pyridinyl, in each case unsubstituted or monosubstituted (preferably with OCH₃, CH₃, OH, SH, CF₃, F, Cl, Br or I); or C₃₋₈ cycloalkyl, especially cyclopropyl, cyclobutyl, cyclopentyl, cyclohexyl or cycloheptyl, in each case unsubstituted or monosubstituted.

In another preferred embodiment, in the amino acids according to Formula I,

one of the residues R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl; or C₄₋₇ cycloalkyl, preferably cyclobutyl, cyclopentyl, cyclohexyl or cycloheptyl, especially cyclobutyl, cyclopentyl or cyclohexyl, in each case unsubstituted or monosubstituted.

More preferably, one of the residues R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C3-10 alkyl, especially n-propyl, i-propyl, n-butyl, i-butyl, sec.-butyl, tert.-butyl, pentyl, hexyl, heptyl or octyl.

In a preferred embodiment of the invention, the amino acids according to the invention are selected from the following group:

2-amino-3-methylheptanoic acid

2-amino-3-methyloctanoic acid

2-amino-3-methylnonanoic acid

2-amino-3-methyldecanoic acid

2-amino-3-ethylhexanoic acid

2-amino-3-methylundecanoic acid

2-amino-3-cyclobutyl-butanoic acid, and

2-amino-3-cyclohexyl-butanoic acid

which may be in the form of their racemates, their pure stereoisomers, especially enantiomers or diastereomers, or in the form of mixtures of the stereoisomers, especially the enantiomers or diastereomers, in any mixing ratio; in the form of their acids or their bases or in the form of their salts, especially the physiologically acceptable salts, or in the form of their solvates, especially the hydrates; preferably the hydrochloride or the sodium salt.

The amino acids according to the invention are toxicologically safe, thus they are suitable as a pharmaceutical active agent in medicaments or pharmaceutical compositions. The invention therefore also provides medicaments containing at least one amino acid according to the invention, as well as optionally suitable additives and/or auxiliary substances and/or optionally further active agents.

The same applies to the amino acids used according to the invention in the said indications, since the amino acids used according to the invention are also toxicologically safe, so that they are suitable as a pharmaceutical active agent in medicaments. The invention therefore also provides medicaments containing at least one of the amino acids used according to the invention, as well as optionally suitable additives and/or auxiliary substances and/or optionally further active agents.

In addition to at least one substituted amino acid according to the invention, the medicaments according to the invention optionally contain suitable additives and/or auxiliary substances, for instance support materials, fillers, solvents, diluents, colorants and/or binders, and may be administered as liquid pharmaceuticals in the form of injection solutions, drops or drinkable liquids, as semi-solid pharmaceuticals in the form of granules, tablets, pellets, patches, capsules, plasters or aerosols. The selection of the auxiliary substances etc., as well as the amounts thereof to be used, depend on whether the medicament is to be applied orally, perorally, parenterally, intravenously, intraperitoneally, intradermally, intramuscularly, intranasally, buccally, rectally or topically, for example onto the skin, the mucous membrane or into the eyes. Preparations in the form of tablets, coated pills, capsules, granules, drops, drinkable liquids and syrups are suitable for oral application, and solutions, suspensions, readily reconstitutable dry preparations as well as sprays are suitable for parenteral, topical and inhalative application. Amino acids according to the invention in a depot, in dissolved form or in a plaster, optionally with the addition of media promoting skin penetration, are suitable percutaneous application preparations. Orally and percutaneously usable preparation forms may release the amino acids according to the invention in a controlled way. In principle, other active agents known to the person skilled in the art may be added to the medicaments according to the invention.

The amount of active agent to be administered to the patient varies according to the patient's weight, the type of application, the indication and the severity of the disease. From 0.005 to 1000 mg/kg, preferably from 0.05 to 5 mg/kg, of at least one amino acid according to the invention is conventionally applied.

In a preferred form of the medicaments according to the invention, an amino acid according to the invention which is contained is present as a pure diastereomer and/or enantiomer, as a racemate or as a non-equimolar or equimolar mixture of the diastereomers and/or enantiomers.

The invention also provides methods using an amino acid according to Formula I according to the invention to manufacture a medicament for treating pain, especially neuropathic, chronic or acute pain, epilepsy and/or migraines,

or

for the manufacture of a medicament for treating hyperalgesia and allodynia, especially thermal hyperalgesia, mechanical hyperalgesia and allodynia and cold allodynia, or inflammatory or postoperative pain

or

for the manufacture of a medicament for treating hot flashes, postmenopausal complaints, amyotrophic lateral sclerosis (ALS), reflex sympathetic dystrophy (RSD), spastic paralysis, restless leg syndrome, acquired nystagmus; psychiatric or neuropathological disorders, such as bipolar disorders, anxiety, panic attacks, mood fluctuations, manic behavior, depression, manic-depressive behavior; painful diabetic neuropathy, symptoms and pain due to multiple sclerosis or Parkinson's disease, neurodegenerative diseases, such as Alzheimer's disease, Huntington's disease, Parkinson's disease and epilepsy; gastric intestinal injury; erythromelalgic or post-poliomeylitic pain, trigeminal or post-therapeutic neuralgia; or as an anticonvulsive, analgesic or anxiolytic.

In each of the aforementioned uses and/or methods, according to the invention, it may be preferable for an amino acid which is used to be present as a pure diastereomer and/or enantiomer, as a racemate or as a non-equimolar or equimolar mixture of the diastereomers and/or enantiomers.

The invention also provides a method of treating a non-human mammal or a human, which or who requires treatment of medically relevant symptoms, by administering a therapeutically effective dose of an amino acid according to the invention, or an amino acid used according to the invention, or a medicament according to the invention. The invention relates especially to corresponding methods of treating pain, especially neuropathic, chronic or acute pain; migraines, hyperalgesia and allodynia, especially thermal hyperalgesia, mechanical hyperalgesia and allodynia and cold allodynia, or inflammatory or postoperative pain; epilepsy, hot flashes, postmenopausal complaints, amyotrophic lateral sclerosis (ALS), reflex sympathetic dystrophy (RSD), spastic paralysis, restless leg syndrome, acquired nystagmus; psychiatric or neuropathological disorders, such as bipolar disorders, anxiety, panic attacks, mood fluctuations, manic behavior, depression, manic-depressive behavior; painful diabetic neuropathy, symptoms and pain due to multiple sclerosis or Parkinson's disease, neurodegenerative diseases, such as Alzheimer's disease, Huntington's disease, Parkinson's disease and epilepsy; erythromelalgic or post-poliomyelitic pain, trigeminal or post-therapeutic neuralgia.

The invention also provides a method of producing an amino acid according to the invention, in a form as described below.

General Method of Preparing the Substituted α Amino Acids

For the synthesis work, the reactions described in the literature are employed, and experience known in house was used.

The invention also provides a method of producing a compound of Formula 1 according to Mechanism 1:

Mechanism 1:

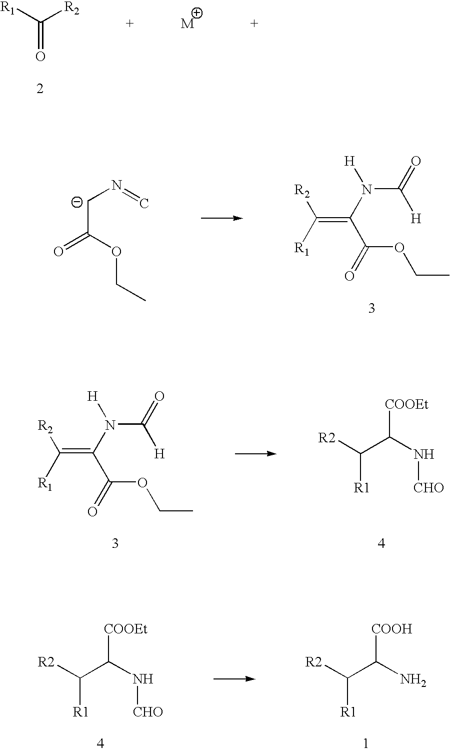

Deprotonation of the ethyl isocyanoacetate with bases such as butyl lithium, sodium hydride or potassium tert.-butylate and subsequent reaction with ketones of Formula 2 in tetrahydrofuran (THF) leads to ethyl (E,Z)-2-formylaminoacrylates of Formula 3. By reaction of ethyl (E,Z)-2-formylaminoacrylates of Formula 3 with Pd/H₂, formylamino ethyl esters of Formula 4 are obtained. Reaction of the formylamino ethyl esters of Formula 4 with hydrochloric acid leads to the amino acids of Formula 1. Diastereomer separation is carried out at a suitable stage by means of HPLC, column chromatography or crystallization. Enantiomer separation is carried out at the final stage likewise by means of HPLC, column chromatography or crystallization. According to this method, the amino acids of Formula 1 are obtained as hydrochlorides. Further salt forms are obtained by base liberation or re-precipitation according to conventional methods well-known in the art.

A method of producing the amino acids according to the invention, with the following steps, is therefore also provided:

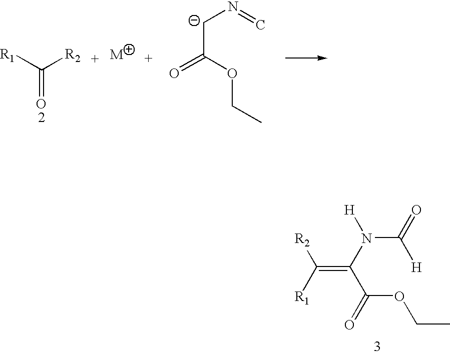

a) deprotonation of the ethyl isocyanoacetate with bases and subsequent reaction with ketones of Formula 2, in which

one of the residues R¹ and R² denotes C₁₋₃ alkyl, especially methyl, ethyl, n-propyl or i-propyl, in each case unsubstituted or mono- or polysubstituted; and the other of the residues R¹ and R² denotes branched or unbranched, saturated or unsaturated, unsubstituted or mono- or polysubstituted C₃₋₁₀ alkyl, especially n-propyl, i-propyl, n-butyl, i-butyl, sec.-butyl, tert.-butyl, pentyl, hexyl, heptyl or octyl; or aryl/heteroaryl, especially phenyl, naphthyl, furanyl, thiophenyl, pyrimidinyl or pyridinyl, in each case unsubstituted or monosubstituted (preferably with OCH₃, CH₃, OH, SH, CF₃, F, Cl, Br or I); or C₃₋₈ cycloalkyl, especially cyclopropyl, cyclobutyl, cyclopentyl, cyclohexyl or cycloheptyl, in each case unsubstituted or monosubstituted;

preferably in tetrahydrofuran leads to ethyl (E,Z)-2-formylaminoacrylates of Formula 3,

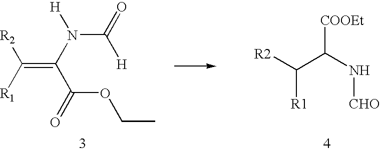

b) reaction of ethyl (E,Z)-2-formylaminoacrylates of Formula 3 with Pd/H₂ leads to formylamino ethyl esters of Formula 4,

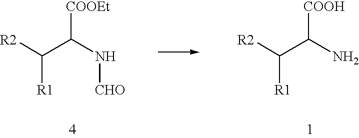

c) reaction of the formylamino ethyl esters of Formula 4 with acids, preferably hydrochloric acid, leads to the amino acids of Formula 1, or Formula I, optionally followed or interrupted by diastereomer separation at a suitable stage by means of HPLC, column chromatography or crystallization, or followed by enantiomer separation by means of HPLC, column chromatography or crystallization.

Salt Formation

The compounds of Formula I can be converted, in the manner well known to those ordinarily skilled in the art, into their salts by using physiologically acceptable acids, for example hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydrolλ⁶-benzo[d]isothiazol-3-one (saccharinic acid), monomethylsebacic acid, 5-oxo-proline, hexane-l-sulfonic acid, nicotinic acid, 2-, 3- or 4-aminobenzoic acid, 2,4,6-trimethyl-benzoic acid, a-lipoic acid, acetylglycine, acetylsalicylic acid, hippuric acid and/or aspartic acid. The salt formation is advantageously carried out in a solvent, for example diethyl ether, diisopropyl ether, alkyl acetate, acetone and/or 2-butanone or alternatively water. Trimethylchlorosilane in aqueous solution is furthermore suitable for producing the hydrochlorides. It is also possible to convert the basic salts by using metal ions, for example: alkali metal and alkaline-earth metal ions.

The invention will be further explained below by examples, but without restricting it to them.

### EXAMPLES

The following examples present compounds according to the invention and their preparation, and the efficacy studies carried out with them.

The following indications generally apply in this context:

The chemicals and solvents used were commercially obtained from the conventional suppliers (Acros, Avocado, Aldrich, Fluka, Lancaster, Maybridge, Merck, Sigma, TCI etc., or synthesized according to methods well-known to those skilled in the art).

Analyses was carried out using ESI mass spectrometry or HPLC.

Syntheses:

### Example 1

Synthesized Compounds:

Representative examples of the compounds used or claimed in the scope of this invention follow:

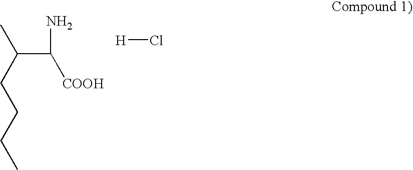

rac-2-amino-3-methyl-heptanoic acid hydrochloride as a 1:1 D/L mixture

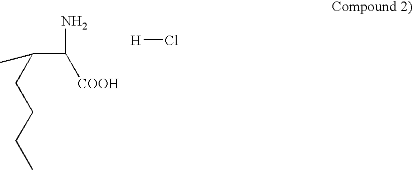

rac-2-amino-3-methyl-octanoic acid hydrochloride as a 1:1 D/L mixture

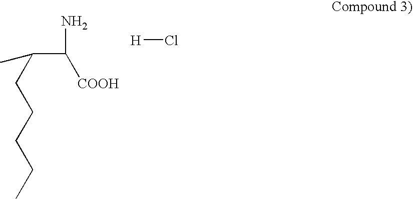

rac-D-2-amino-3-methyl-octanoic acid hydrochloride

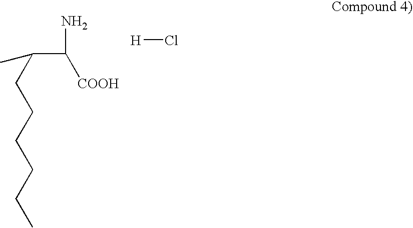

rac-D-2-amino-3-methyl-nonanoic acid hydrochloride

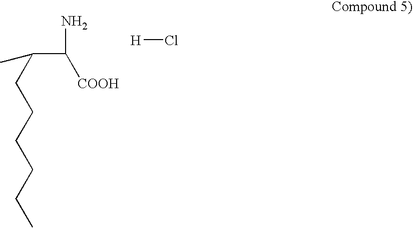

rac-L-2-amino-3-methyl-nonanoic acid hydrochloride

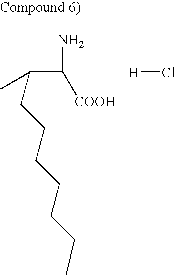

rac-2-amino-3-methyl-decanoic acid hydrochloride as a 1:1 D/L mixture

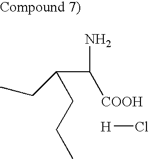

rac-amino-3-ethyl-hexanoic acid hydrochloride as a 1:1 D/L mixture

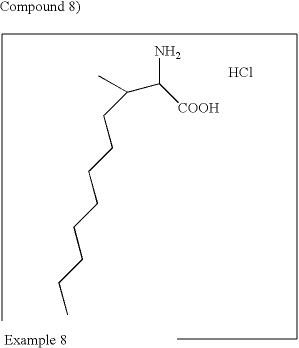

rac-2-amino-3-methyl-undecanoic acid hydrochloride as a 1:1 D/L mixture

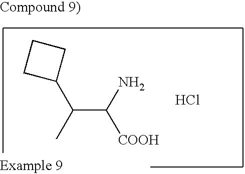

rac-2-Amino-3-cyclobutyl-butanoic acid hydrochloride as a 1:1 D/L mixture

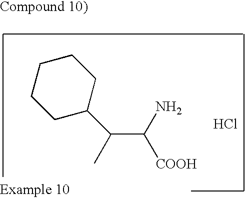

rac-2-amino-3-cyclohexyl-butanoic acid hydrochloride as a 1:1 D/L mixture

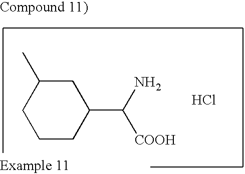

rac-amino-(3-methyl-cyclohexyl)-ethanoic acid hydrochloride

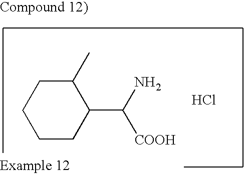

rac-amino-(2-methyl-cyclohexyl)-ethanoic acid hydrochloride

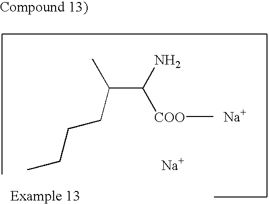

rac-2-amino-3-methyl-heptanoic acid sodium salt as a 1:1 D/L mixture (Na salt of Compound 1);

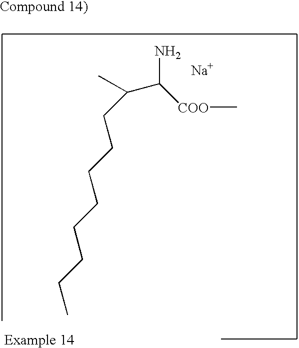

rac-2-amino-3-methyl-decanoie acid sodium salt as a 1:1 D/L mixture (Na salt of Compound 6);

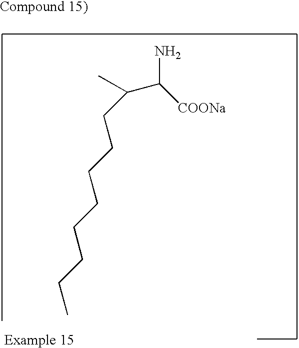

rac-2-amino-3-methyl-undecanoic acid sodium salt as a 1:1 D/L mixture (Na salt of Compound 8);

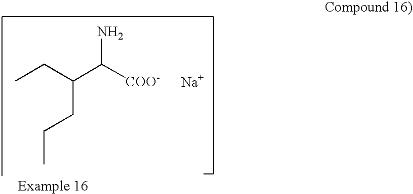

rac-2-amino-3-ethyl-hexanoic acid sodium salt as a 1:1 D/L mixture (Na salt of Compound 7);

### Example 2

Method of Preparing Compounds According to the Invention

The following examples serve to explain the method according to the invention in more detail.

The yields of the compounds are not optimized.

All temperatures are uncorrected.

Silica Gel 60 (0.040-0.063 mm) from E. Merck, Darmstadt, was used as the stationary phase for the column chromatography.

The thin-layer chromatography studies were carried out using HPTLC pre-coated plates, Silica Gel 60 F 254 from E. Merck, Darmstadt.

The mixing ratios of the eluents for all chromatography studies are always indicated in volume/volume.

The reference ether means diethyl ether.

Unless otherwise indicated, petroleum ether with the boiling range 50° C.-70° C. was used.

Procedure 1

Preparation of Compound 6 (Prod. 1):

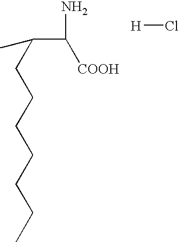

rac-2-amino-3-methyl-decanoic acid hydrochloride as a 1:1 D/L mixture (Compound 6=Prod. 1);

1. Glycine ethyl ester hydrochloride (Prod. 2)

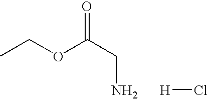

247.3 g of thionyl chloride and 130 g of glycine were introduced into 1000 ml of ethanol at −10° C. After removing the ice bath, a further equivalent of glycine was added portion-wise. The mixture was then stirred under reflux for 2 h. After cooling to room temperature, the excess alcohol and the thionyl chloride were removed using a rotary evaporator. The white solid obtained was treated twice with ethanol, and the latter was in turn removed using the rotary evaporator, in order to completely remove any adhering thionyl chloride. After re-crystallization from ethanol, 218.6 g (90.4% of theor.) of the title compound (Prod. 2) was obtained.

2. Ethyl formylaminoacetate (Prod. 3)

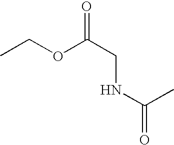

218 g of glycine ethyl ester hydrochloride (Prod. 2) were suspended in 1340 ml of ethyl formate. 223 mg of toluenesulfonic acid were added, and the mixture was heated to reflux. 178 g of triethylamine were then added drop-wise to the boiling solution, and the reaction solution was stirred overnight under reflux. After cooling to RT, the precipitated ammonium chloride salt was filtered off, the filtrate was concentrated to about 20% of the original volume and cooled to −5° C. The newly precipitated ammonium chloride salt was filtered off, the filtrate was concentrated again and distilled at 1 mbar. 184 g (90.3% of theor.) of the title compound (Prod. 3) were obtained in this way.

3. Ethyl isocyanoacetate (Prod. 4)

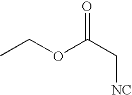

50 g of ethyl formylaminoacetate (Prod. 3) and 104 g of diisopropylamine were introduced into 400 ml of dichloromethane and cooled to −3° C. 70.1 g of phosphoryl chloride in 400 ml of dichloromethane were then added drop-wise, and stirring was subsequently carried out for a further hour at this temperature. After the ice bath had been removed and room temperature had been reached, hydrolysis was carefully carried out with 400 ml of 20% strength sodium carbonate solution. After stirring for 60 minutes at RT, 400 ml of water and then 200 ml of dichloromethane were added. The phases were separated, and the organic phase was washed twice, each time with 100 ml of 5% of Na₂CO₃ solution, and dried over MgSO₄. The solvent was evaporated in a rotary evaporator, and the remaining brown oil was distilled. 34.16 g (79.3% of theor.) of the title compound (Prod. 4) were obtained in this way.

4. Ethyl (E)/(Z)-2-formylamino-3-methyldec-2-enoate (Prod. 5)

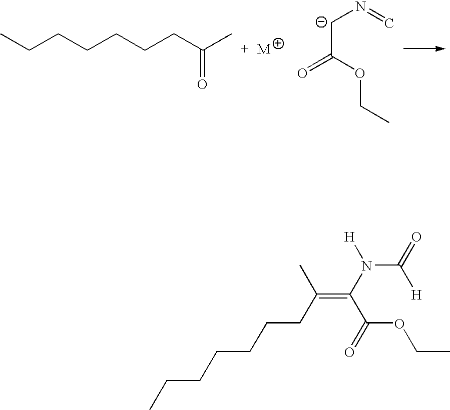

A solution of 22 g of ethyl isocyanoacetate (Prod. 4) in 49 ml of THF was added drop-wise to a suspension of 23 g of potassium tert.-butylate 148 ml of THF at from −70° C. to −60° C. while stirring. Stirring was continued for 20 min; 27.7 g of 2-nonanone in 24 ml of THF were subsequently added drop-wise at this temperature. After warming to RT, 11.7 ml of glacial acetic acid were added. 15 minutes after adding the glacial acetic acid (TLC control: ether:hexane 4:1), the solvent was evaporated. The residue was treated with 300 ml of diethyl ether and 200 ml of water. The organic phase was separated off and the aqueous phase was washed twice, each time with 120 ml of ether. The combined organic phases were washed with 80 ml of 2N NaHCO₃ solution and dried over MgSO₄. The solvent was subsequently evaporated. The raw product obtained in this way was digested with 200 n-hexane. The solid was filtered off, washed four times, in each case with 80 ml of hexane, and dried in an oil-pump vacuum. 34.8 g (69.9% of theor.) of ethyl (E) and (Z)-2-formylamino-3-methyldec-2-enoate (Prod. 5) (E/Z ratio: 1:1) were obtained in this way as a white solid.

5. Ethyl 2-formylamino-3-methyl-decanoate as a 1:1 D/L mixture (Prod. 6)

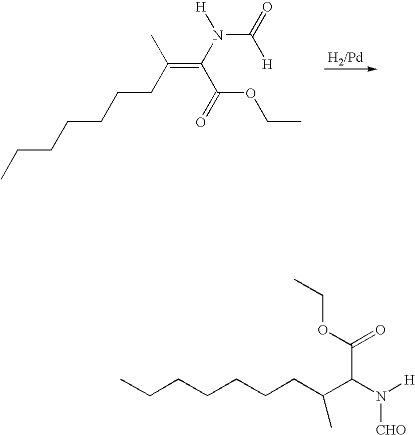

5 g of ethyl (E)/(Z)-2-formylamino-3-methyldec-2-enoate (Prod. 5) (E/Z ratio: 1:1) were dissolved in 100 ml of methanol at RT under a nitrogen atmosphere, and subsequently treated with 0.25 g of Pd-C (5% strength). The mixture was subsequently hydrogenated under a hydrogen atmosphere. After completion of the hydrogenation (TLC control:ether:hexane 4:1), the batch was vacuum-pumped through 50 ml of filter earth and the filter earth was washed with methanol. The solvent was removed from the organic phase. 5.1 g (86% of theor.) of ethyl 2-formylamino-3-methyl-decanoate as a 1:1 D/L mixture (Prod. 6) were obtained in this way.

6. rac-2-amino-3-methyl-decanoic acid hydrochloride as a 1:1 D/L mixture (Compound 6=Prod. 1)

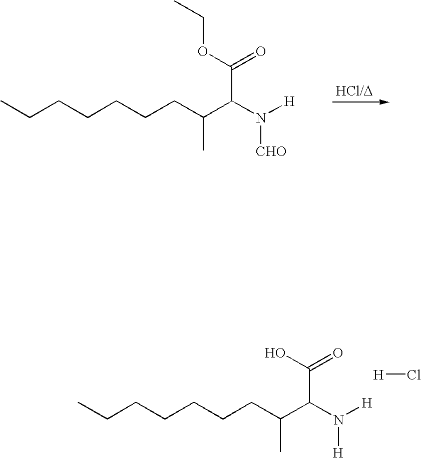

5 g of ethyl 2-formylamino-3-methyl-decanoate as a 1:1 D/L mixture (6) were added to 300 ml of 6N hydrochloric acid at RT and subsequently stirred for 24 h under reflux (TLC control: dichloromethane : methanol : glacial acetic acid: 35:5:3). After cooling to RT, further stirring was carried out while cooling with ice. The precipitated white solid was vacuum-pumped, washed with ether and subsequently dried in a vacuum. 4.2 g (94.9% of theor.) of rac-2-amino-3-methyl-decanoic acid hydrochloride as a 1:1 D/L mixture (Compound 6=Prod. 1) were obtained in this way.

Procedure 2:

Preparation of Compound 1 (Prod. 7)

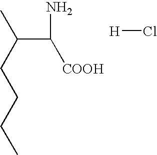

rac-2-amino-3-methyl-heptanoic acid hydrochloride as a 1:1 D/L mixture (Compound 1=Prod. 7)

rac-2-amino-3-methyl-heptanoic acid hydrochloride as a 1:1 D/L mixture (Compound 1=Prod. 7) was obtained by using 2-hexanone instead of 2-nonanone in Procedure 1.

Procedure 3:

Preparation of Compound 2 (Prod. 8)

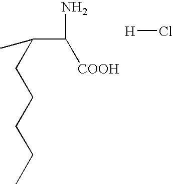

rac-2-amino-3-methyl-octanoic acid hydrochloride as a 1:1 D/L mixture (Compound 2=Prod. 8)

rac-2-amino-3-methyl-octanoic acid hydrochloride as a 1:1 D/L mixture (Compound 2=Prod. 8) was obtained by using 2-heptanone instead of 2-nonanone in Procedure 1.

Procedure 4: Preparation of Compound 7 (Prod. 9)

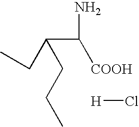

rac-amino-3-ethyl-hexanoic acid hydrochloride as a 1:1 D/L mixture (Compound 7=Prod. 9)

rac-amino-3-ethyl-hexanoic acid hydrochloride as a 1:1 D/L mixture (Compound 7=Prod. 9) was obtained by using 3-hexanone instead of 2-nonanone in Procedure 1.

Procedure 5:

Preparation of Compound 3 (Prod. 10)

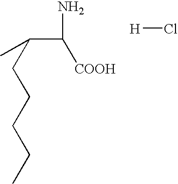

rac-D-2-amino-3-methyl-octanoic acid hydrochloride (Compound 3=Prod. 10)

The process corresponded to Procedure 1; parts 1, 2, 3, 4; except that the 2-nonanone used in Procedure 1 was replaced here by 2-heptanone. Differences arose starting from Procedure 1 part 5.

5) Ethyl D-2-formylamino-3-methyl-octanoate (Prod. 11)

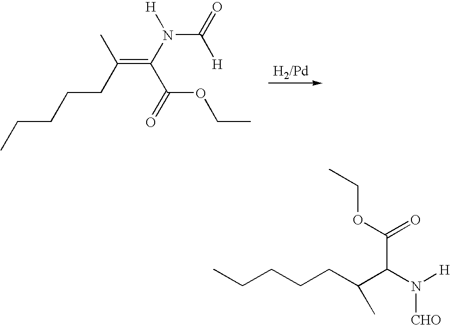

5 g of ethyl (E)/(Z)-2-formylamino-3-methyloct-2-enoate (Prod. 12) (E/Z ratio: 1:1) were dissolved in 100 ml of methanol at RT under a nitrogen atmosphere, and subsequently treated with 0.25 g of Pd—C (5% strength). The mixture was subsequently hydrogenated under a hydrogen atmosphere. After completion of the hydrogenation (TLC control: ether :hexane 4:1), the batch was vacuum-pumped through 50 ml of filter earth and the filter earth was washed with methanol. The solvent was removed from the organic phase, which was chromatographed on silica gel with ether/hexane (4:1). 2.2 g (40% of theor.) of ethyl D-2-formylamino-3-methyl-octanoate (Prod. 11) were obtained as the first fraction.

6. rac-D-2-amino-3-methyl-octanoic acid hydrochloride (Compound 3=Prod. 10)

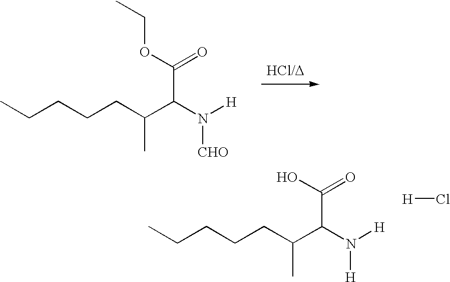

2.2 g of ethyl D-2-formylamino-3-methyl-octanoate (Prod. 11) were added to 300 ml of 6N hydrochloric acid at RT and subsequently stirred for 24 h under reflux (TLC control:dichloromethane:methanol:glacial acetic acid: 35:5:3). After cooling to RT, further stirring was carried out while cooling with ice. The precipitated white solid was vacuum-pumped, washed with ether and subsequently dried in a vacuum. 2 g (90% of theor.) of rac-D-2-amino-3-methyl-octanoic acid hydrochloride (Compound 3=Prod. 10) were obtained in this way.

Procedure 7:

Preparation of Compound 4 (Prod. 13) and Compound 5 (Prod. 14)

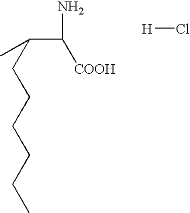

The process corresponded to Procedure 1; parts 1, 2, 3, 4; except that the 2-nonanone used in Procedure 1 was replaced here by 2-octanone. Differences arose starting from Procedure 1 part 5.

5) Ethyl D-2-formylamino-3-methyl-nonanoate (Prod. 15) and ethyl L-2-formylamino-3-methyl-nonanoate (Prod. 16)

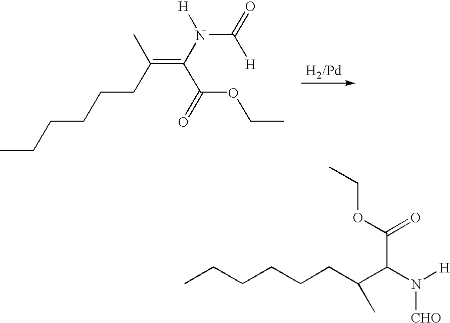

5 g of ethyl (E)/(Z)-2-formylamino-3-methylnon-2-enoate (Prod. 17) (E/Z ratio: 1:1) were dissolved in 100 ml of methanol at RT under a nitrogen atmosphere, and subsequently treated with 0.25 g of Pd—C (5% strength). The mixture was subsequently hydrogenated under a hydrogen atmosphere. After completion of the hydrogenation (TLC control:ether:hexane 4:1), the batch was vacuum-pumped through 50 ml of filter earth and the filter earth was washed with methanol. The solvent was removed from the organic phase, which was chromatographed on silica gel with ether/hexane (4:1). 2.2 g (40% of theor.) of ethyl D-2-formylamino-3-methyl-nonanoate (Prod. 15) as the first fraction, and 1 g (22% of theor.) of ethyl L-2-formylamino-3-methyl-nonanoate (Prod. 16) as the second fraction, were obtained.

6. rac-D-2-amino-3-methyl-nonanoic acid hydrochloride (Compound 4=Prod. 13) and rac-L-2-amino-3-methyl-nonanoic acid hydrochloride (Compound 5=Prod. 14)

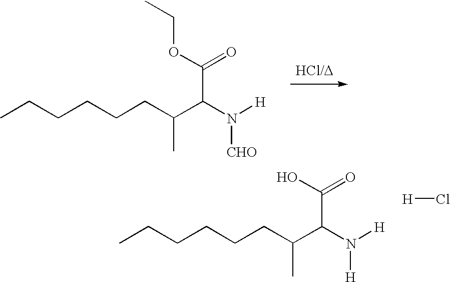

1 g of ethyl D-2-formylamino-3-methyl-nonanoate (Prod. 15), and 1 g of ethyl L-2-formylamino-3-methyl-nonanoate (Prod. 16), respectively, were added to 150 ml of 6N hydrochloric acid at RT and subsequently stirred for 24 h under reflux (TLC control:dichloromethane:methanol:glacial acetic acid: 35:5:3). After cooling to RT, further stirring was carried out while cooling with ice. The precipitated white solid was vacuum-pumped, washed with ether and subsequently dried in a vacuum. 0.9 g (90% of theor.) of rac-D-2-amino-3-methyl-nonanoic acid hydrochloride (Compound 4=Prod. 13), and 0.9 g (90% of theor.) of rac-L-2-amino-3-methyl-nonanoic acid hydrochloride (Compound 5=Prod. 14), respectively, were obtained in this way.

Pharmacological Studies

### Example 3

Binding Assay

In the binding assay, gabapentin is used in order to check the binding and affinities of the selected compounds. The affinity of the compounds according to the invention was measured by using the displacement of gabapentin from its binding site. If the selected compounds can displace gabapentin from its binding site, then it may be expected they will present comparable pharmacological properties to gabapentin, for example as an agent against pain or epilepsy. The compounds according to the invention exhibit good inhibition/displacement of gabapentin in this assay. The studied compounds therefore exhibit an affinity with the hitherto unknown gabapentin binding site in this biochemical assay.

For gabapentin, an IC₅₀ value of 60 nM was determined. Some of the synthesized compounds of Formula 1 exhibit a significantly better affinity than the comparison substance gabapentin (see Table 1).

TABLE 1

| Compound No. | Affinity (IC₅₀) nM |
|---|---|
| 1 | 202 |
| 2 | 50 |
| 3 | 88 |
| 4 | 35 |
| 5 | 29 |
| 6 | 30 |
| 7 | 315 |
| 8 | 151 |
| 9 | 115 |
| 10 | 90 |
| 11 | 35 |
| 12 | 66 |
| 13 | 77 |
| 14 | 21 |
| 15 | 14 |
| 16 | 160 |
| Gabapentin | 60 |

### Example 4

Analgesia Trial Using the Writhing Test on Mice

The anti-nociceptive efficacy of the compounds according to the invention was studied on mice with the phenylquinone-induced writhing test, modified after I. C. Hendershot, J. Forsaith, J. Pharmacol. Exp. Ther. 125, 237-240 (1959). Male NMRI mice with a weight of from 25-30 g were used for this purpose. 10 minutes after intravenous administration of a compound according to the invention, groups of 10 animals per substance dose were intraperitoneally given 0.3 ml/mouse of a 0.02% strength aqueous solution of phenylquinone (phenylbenzoquinone, Sigma, Deisenhofen; production of the solution with the addition of 5% ethanol and storage in a water bath at 45° C.). The animals were placed individually in observation cages. The number of pain-induced stretching movements (so-called writhing reactions =straightening of the body with stretching of the hind extremities) were counted by means of a push-button counter 5-20 minutes after the phenylquinone administration. Animals which received physiological saline solution i.v. and phenylquinone i.v. were also included as a control.

All the substances were tested in the standard dose of 10 mg/kg. The percentage inhibition (% inhibition) of the writhing reactions by a substance was calculated according to the following formula:

% inhibition=100−[WR of treated animals/ WR of control×100]

All the compounds according to the invention which were studied exhibited an effect in the writhing test.

The results of selected writhing studies are collated in Table 2. Gabapentin exhibited an ED₅₀ of 38 mg/kg.

TABLE 2
analgesia trial using the writhing test on mice

| compound No | Writhing mouse i.v. ED₅₀ |
|---|---|
| 2 | 28 mg/kg |
| gabapentin | 38 mg/kg |

### Example 5

Microiontophoresis on Anaesthetized Rats

(derivation of evoked ascending potentials of individual cells in the dorsal horn of the spinal cord of anaesthetized rats after microiontophoretic application of excitatory amino acids (EAAs))

Male rats (Sprague Dawley, Janvier) with a body weight of from 280 g to 350 g were used. To induce the anaesthesia, inhalation anaesthetization was carried out with 4.0-5.0% halothane in a mixture of 200 ml/min oxygen (O₂) and 400 ml/min dinitrogen monoxide (N₂O) in a plexiglas anaesthetization box. During the preparatory phase, the halothane concentration was reduced to 1.0-1.5%. For the rest of the experiment, the inhalation anaesthetization was then replaced by bolus application of 40 mg/kg of chloralose in 3.5% Haemaccel solution (i.v.) and maintained by continuous infusion of 20 mg/kg/2.0 ml/h of chloralose. After the induction anaesthetization (4.0-5.0% halothane, see above), the tracheal tube was fitted through a syringe cylinder (20 ml Omnifix, Luer) shortened to about 20 mm, which was placed over the mouth and nose of the animals, with a 2.5% halothane concentration. The halothane concentration was then reduced to about 1.5%. Exhaled air was extracted through a closed system. For the application of test substances, a PE-20 (1.09*0.38mm) catheter was fitted into the dorsal branch of the left V. jugularis. A PE-50 tube (0.9569 mm*0.58 mm) was inserted into the left arteria carotis as a catheter for continuously monitoring the blood pressure. For the laminectomy, the animals were then placed in the prone position. An incision was made along the midline from the nape of the neck to the pelvic region. A blood-pressure drop frequently observed following this was treated by local application of lidocaine HCl with adrenaline (xylocitin 2% with adrenaline 0.001%, Jenapharm). The superficial tissue layers on both sides of the spinal column were removed in the vicinity of the vertebral bodies from sacral 2 (S2) to thoracic 8 (Th8). The musculature associated with the vertebral bodies was also removed by careful scraping with a scalpel. The actual laminectomy begins at vertebral body L2 (lumbar 2), after removing the processus spinalis from vertebra L3, and extends into the TH8 rostral region as far as where the large dorsal veins branch off laterally and caudally. The bones of the individual vertebral bodies were carefully removed in small stages by means of fine rongeurs. The musculature around TH8/9 and L1/S2 was also removed, so that immobilization clamps could be fitted later. Exposed tissue was protected against drying by applied 0.9% strength NaCI solution or thin PVC strips. The temperature was controlled between 36.5° C. and 37.5° C. for all the animals, from the start of the catheterization until the end of the experiment, using an electronically regulated heating pad and a rectal thermal sensor (Harvard Homeothermic Blanket System). After laminectomy had been carried out, the animals were transferred into a fixation frame constructed in-house. The horizontal fastening was carried out using modified clamps (Harvard General Purpose Clamp) in the region of T8/9 and S2. The lateral fixation was carried out using side clamps in the region of the intended lead-off (segment L5/6). A pool was formed using skin which was lifted up and fastened to the fixation frame using threads. The dura mater was removed over the entire exposed region, and the spinal cord was covered with fluid paraffin oil (pool). After changing over from halothane to chloralose, the animals spontaneously breathed additionally oxygen-enriched room air (200 ml/min). The blood pressure was continuously displayed on the screen (Spike 2, Cambridge Electronic) via the arterial catheter, which was connected to a pressure transmitter (Elcomatic EM751A, filled with paraffin oil), and to the blood-pressure preamplifier NL108 (Neurolog). The systolic pressure was in this case intended to be close to 100 mmHg or above. In addition to the blood pressure, the local blood circulation was visually assessed, a pink skin color of the paws indicating normal microcirculation. An intact blood supply of the spinal cord is manifested by a cherry-red dorsal vein and fast blood flow into the smaller veins. Multibarrel glass microelectrodes made in-house were used to lead off action potentials and to eject excitatory amino acids (EAAs). Using shrinkable tubing (Shrink-KON HSB 250 6.4 to 3.2 mm; RS order No. RS 208-9005), the outer angled-off capillaries were temporarily fastened so that the central capillary protrudes about 35 mm. The permanent fixation is then carried out using quick-setting epoxy adhesive (RS Quick Set Epoxy Adhesive RS 850-940). These electrode pre-forms were processed further. A usable electrode has a diameter of 15-20 μm at a distance of about 100 μm from the tip. Before filling the individual capillaries, the tips of the electrodes were broken back to a diameter of 3-5 μm under microscopic observation (Olympus BH-2 microscope; Zeiss measurement eyepiece; magnification×20;×40). The electrode tips were in this case brought into the immediate vicinity (mirror image of the electrode visible) of a glass rod, and broken by carefully touching fine-adjustment screws of the mechanical xy stage. The electrodes were filled using 1 ml Omnifix-F (B. Braun) single-use syringes with Microfil MF34G syringe capillaries (WPI), and stored in the refrigerator (4-6° C.) between the experiments in a frame made in-house. Good electrodes can readily be used several times after appropriate checking. The individual capillaries (barrels) were color-coded with a permanent felt-tip pen and correspondingly filled.

| | |
|---|---|
| Barrel 1: mark: red; NMDA | 100 mM in 100 mM NaCl; pH 7.5-8.0 |
| Barrel 2: mark: green; AMPA | 10 mM in 200 mM NaCl; pH 7.5-8.0 |
| Barrel 3: mark: blue; kainate | 5 mM in 200 mM NaCl; pH 7.5-8.0 |
| Barrel 4: mark: none; current | 150 mM NaCl |
| balance | |
| Barrel 5: mark: none; | 3.5 M NaCl |
| extracellular lead | |

All the barrels were sealed with fluid paraffin oil (slightly dyed with Sudan black) for protection against drying. Before use, the electrical resistance of the central capillary is measured in NaCl solution against a silver-silver chloride pellet, and should be between 1 and 5 mOhm (measuring instrument: Multimeter Voltcraft 4550B; measurement range 20 mOhm). It was found practicable not to determine the resistances of the outer barrels until after insertion of the electrodes (depth about 200-400 μm) into the spinal cord of the test animal, the IP-2 microiontophoresis pump being used. Usable values are about 20-100 mOhm. Electrodes with resistances which are too high (especially the central capillary) can be broken back further under microscopic observation and re-checked.

When gabapentin is studied in this test model, this substance exhibits dose-dependent and selective inhibition of the AMPA response of spinal neurons in the dorsal horn in the spinal cord of anaesthetized rats. The ED50 was 106 mg/kg. Gabapentin has no affinity for the AMPA receptor. Identical behavior was observable for Compound 16. This compound also exhibits selective inhibition of the AMPA response without having any AMPA affinity. The ED50 was 60 mg/kg.

The following literature gives a broadened overview of the experimental method, and is herein incorporated by reference in its entirety:

Chizh B A, Cumberbatch M J, Herrero J F, Stirk G C, Headley P M. Stimulus intensity, cell excitation and the N-methyl-D-aspartate receptor component of sensory responses in the rat spinal cord in vivo. Neuroscience. September; 1997 80(1): 251-65; and

Chizh B A, Headley P M. Thyrotropin-releasing hormone (TRH)-induced facilitation of spinal neurotransmission: a role for NMDA receptors. Neuropharmacology. January; 1994 33(1): 115-21.

### Example 6

Mechanical Hyperalgesia After Paw Incision on Rats (Paw Incision Model)

1. Introduction

In this model, the pain due to a wound in the vicinity of an incision on the plantar side of a hind paw of the rat is studied as a model of postoperative pain (Brennan, T. J., Vandermeulen, E. P., Gebhart, G. F., Pain (1996) 493-501). For this purpose, the retraction latency after point mechanical stimulation with an electronic von Frey filament is determined. After the paw incision, a mechanical hyperalgesia develops and remains stable over several days.

2. Material and methods

Paw Incision:

Male Sprague Dawley rats (body weight 200-300 g) are used. Under halothane anaesthetization, a 1 cm long incision, starting 0.5 cm from the proximal end of the heel, is made through the skin, fascia and M. plantaris, and is closed with two sutures.

3. Test Procedure:

By using an electronic von Frey filament (Digital Transducer Indicator Model 1601C, IITC Inc.), the retraction threshold of the paw, expressed in grams, after point mechanical stimulation is determined. For this purpose, the retraction threshold is measured five times per measurement point at an interval of 30 sec, and the individual median is determined, with the aid of which the average of the animal population is calculated. 10 rats are tested per test animal group.

To study the primary hyperalgesia, the retraction threshold on the ipsilateral paw is determined in the immediate vicinity of the incision, and also in the same position on the contralateral paw. The measurements are taken twice before the operative intervention in order to determine the pre-test average, postoperatively immediately before the substance administration and at various times after substance administration (as a rule 15, 30, 60, 90, 120 min p. appl.). The studies can be carried out on substances in a period of from 2 hours up to 3 days postoperatively.

4. Evaluation:

The Efficacy of a Substance is Described Using the Effect on the Retraction Threshold of the Ipsilateral Paw:

% MPE=100−[(WTH_{sub}−WTHₚᵣₑ₋ₒₚ)/(WTHₚₒₛₜ₋ₒₚ−WTHₚᵣₑ₋ₒₚ)*100]

MPE: Maximum Possible Effect

WTH_{sub}: retraction threshold after substance administration

WTHₚᵣₑ₋ₒₚ: retraction threshold before the operation (pre-test average)

WTHₚₒₛₜ₋ₒₚ: retraction threshold after the operation and before the substance administration

The Mann-Whitney U test is used for the significance calculation (p<0.05). For dose-dependent effects, the ED₅₀ value is determined with the aid of a regression analysis.

Results:

The results are collated in Table 3:

TABLE 3
Analgesia trial with paw incision on rats

| Compound No | Value |
|---|---|
| 7 | 30% MPE (464 mg/kg i.p.) |
| gabapentin | 66% MPE (100 mg/kg) |

### Example 7

Parenteral Application Form.

38.5 g of Compound 7 are dissolved in 1 l of water for injection purposes at room temperature, and subsequently adjusted to isotonic conditions by adding anhydrous glucose for injection purposes.

What is claimed is:

1. A pharmaceutical composition for treating pain, comprising an effective pain-treating amount of a compound of Formula I 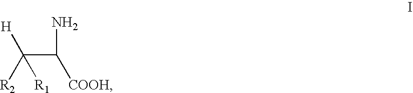
wherein in Formula I
one of R¹ and R² denotes C₁₋₃ alkyl, in each case unsubstituted or mono- or polysubstituted; and the other of R¹ and R² denotes branched or unbranched, saturated or unsaturated, unsubstituted or mono- or polysubstituted C₃₋₁₀ alkyl; or aryl or heteroaryl, unsubstituted or monosubstituted; or unsubstituted or mono substituted C₃₋₈ cycloalkyl;
or
R¹ and R² together denote substituted or unsubstituted (CH₂)₅, so that a substituted or unsubstituted cyclohexyl is obtained, and
a pharmaceutically acceptable excipient.

2. A pharmaceutical composition according to claim 1, wherein the compound of formula I is in the form of a racemate, a pure enantiomer, a pure diastereomer, a mixture of enantiomers in any mixing ratio, a mixture of diastereomers in any mixing ratio, a physiologically acceptable salt, a base, an acid, or a solvate.

3. A pharmaceutical composition according to claim 1, wherein the pharmaceutical composition is for treating neuropathic pain, chronic pain, acute pain, migraine, inflammatory pain, postoperative pain, or pain due to multiple sclerosis or Parkinson's disease.

4. A pharmaceutical composition for treating hyperalgesia, allodynia, hot flash, postmenopausal complaint, amyotrophic lateral sclerosis (ALS), reflex sympathetic dystrophy (RSD), spastic paralysis, restless leg syndrome, acquired nystagmus; a psychiatric or neuropathological disorders; painful diabetic neuropathy, a symptom due to multiple sclerosis or Parkinson's disease, a neurodegenerative disease; a gastric intestinal injury; erythromelalgic or post-poliomyelitic pain, trigeminal or post-therapeutic neuralgia; or for anticonvulsive or anxiolytic treatment,
wherein the pharmaceutical composition comprises a pharmaceutically acceptable excipient and an effective amount of a compound of Formula I, 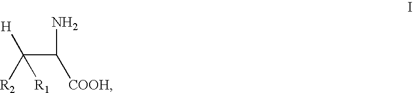
in which
one of R¹ and R² denotes C₁₋₃ alkyl, in each case unsubstituted or mono- or polysubstituted; and the other of R¹ and R² denotes branched or unbranched, saturated or unsaturated, unsubstituted or mono- or polysubstituted C₃₋₁₀ alkyl; or aryl, or heteroaryl, unsubstituted or monosubstituted; or unsubstituted or monosubstituted C₃₋₈ cycloalkyl;
or
R¹ and R² together denote substituted or unsubstituted (CH₂)₅, so that a substituted or unsubstituted cyclohexyl is obtained.

5. A pharmaceutical composition according to claim 4, which is for the treatment of thermal hyperalgesia, mechanical hyperalgesia, cold allodynia, bipolar disorder, anxiety, panic attack, mood fluctuation, manic behavior, depression, manic-depressive behavior; Alzheimer's disease, Huntington's disease, Parkinson's disease and epilepsy.

6. A pharmaceutical composition according to claim 1, wherein
one of R¹ and R² denotes C₁₋₃ alkyl, in each case unsubstituted or mono- or polysubstituted; and the other of R¹ and R² denotes branched or unbranched, saturated or unsaturated, unsubstituted or mono- or polysubstituted C₃₋₁₀ alkyl; aryl or heteroaryl, in each case unsubstituted or monosubstituted; or C₃₋₈ cycloalkyl, in each case unsubstituted or mono substituted.

7. A pharmaceutical composition according to claim 6, wherein
one of the residues R¹ and R² denotes methyl, ethyl, n-propyl or i-propyl, in each case unsubstituted or mono- or polysubstituted; and the other of the residues R¹ and R² denotes n-propyl, i-propyl, n-butyl, i-butyl, sec.-butyl, tert.-butyl, pentyl, hexyl, heptyl, octyl, cyclobutyl, cyclopentyl, cyclohexyl, cycloheptyl, in each case unsubstituted or mono substituted.

8. A pharmaceutical composition according to claim 6, wherein one of R¹ and R² denotes an aryl or a heteroaryl substituted with one or more of OCH₃, CH₃, OH, SH, CF₃, F, Cl, Br and I.

9. A pharmaceutical composition according to claim 1, wherein
R¹ and R² together denote substituted or unsubstituted (CH₂)₅, so that a monosubstituted or unsubstituted cyclohexyl is obtained.

10. A pharmaceutical composotion according to claim 9, wherein
R¹ and R² together denote an unsubstituted (CH₂)₅, so that a unsubstituted cyclohexyl is obtained.

11. A pharmaceutical composition according to claim 9, wherein
R¹ and R² together denote methyl-substituted (CH₂)₅, so that a methyl-substituted cyclohexyl is obtained.

12. A pharmaceutical composition according to claim 1, wherein the compound according to formula I is 2-amino-3-methylheptanoic acid; 2-amino-3-methyloctanoic acid, 2-amino-3-methylnonanoic acid, 2-amino-3-methyldecanoic acid, 2-amino-3-ethylhexanoic acid, 2-amino-3-methylundecanoic acid, 2-amino-3-cyclobutyl-butanoic acid, 2-amino-3-cyclohexyl-butanoic acid, amino-(3-methyl-cyclohexyl)ethanoic acid, or amino-(2-methyl-cyclohexyl)ethanoic acid.

13. A anticonvulsive or anxiolytic method, or a method for treating a disease or a symptom, wherein the disease or symptom is selected from the group consisting of pain, migraine, hyperalgesia, allodynia, hot flash, postmenopausal complaint, amyotrophic lateral sclerosis (ALS), reflex sympathetic dystrophy (RSD), spastic paralysis, restless leg syndrome, acquired nystagmus; a psychiatric or neuropathological disorder, painful diabetic neuropathy, a symptom and pain due to multiple sclerosis or Parkinson's disease, a neurodegenerative disease, gastric intestinal injury; trigeminal, and post-therapeutic neuralgia, the method comprising administering an effective amount of a pharmaceutical composition according to claim 1 to a patient in need thereof.

14. A method according to claim 13, wherein the disease or symptom is selected from the group consisting of neuropathic pain, chronic pain, acute pain, inflammatory pain, postoperative pain, erythromelalgic pain, post-poliomyelitic pain, hyperalgesia, mechanical hyperalgesia, allodynia, cold allodynia, a bipolar disorder, anxiety, panic attack, mood fluctuation, manic behavior, depression, manic-depressive behavior, Alzheimer's disease, Huntington's disease, Parkinson's disease, and epilepsy.

15. A compound selected from the group consisting of 2-amino-3-methyldecanoic acid, 2-amino-3-methylundecanoic acid, 2-amino-3-cyclobutyl-butanoic acid, and 2-amino-3-cyclohexyl-butanoic acid.

16. A compound according to claim 15, wherein the compound is in the form of a racemate, a pure enantiomer, a pure diastereomer, a mixture of a enantiomer in any mixing ratio, or a mixture of a diastereomer in any mixing ratio.

17. A compound according to claim 15, wherein the compound is in the form of an acid, a base, a salt, or a solvate.

18. A compound according to claim 17, wherein the compound is a physiologically acceptable salt, or in the form of a hydrate.

19. A compound according to claim 18, wherein the compound is a hydrochloride or a sodium salt.

20. A pharmaceutical composition comprising a compound of claim 15, and a pharmaceutically acceptable excipient.

21. A anticonvulsive or anxiolytic method, or a method for treating a disease or a symptom, wherein the disease or symptom is selected from the group consisting of pain, migraine, hyperalgesia, allodynia, hot flash, postmenopausal complaint, amyotrophic lateral sclerosis (ALS), reflex sympathetic dystrophy (RSD), spastic paralysis, restless leg syndrome, acquired nystagmus; a psychiatric or neuropathological disorders, painful diabetic neuropathy, a symptom and pain due to multiple sclerosis or Parkinson's disease, neurodegenerative diseases, gastric intestinal injury; trigeminal, and post-therapeutic neuralgia, the method comprising administering an effective amount of a pharmaceutical composition according to claim 20 to a patient in need thereof.

22. A method for according to claim 21, wherein the disease or symptom is selected from the group consisting of neuropathic pain, chronic pain, acute pain, inflammatory pain, postoperative pain, erythromelalgic pain, post-poliomyelitic pain, hyperalgesia, mechanical hyperalgesia, cold allodynia, a bipolar disorder, anxiety, panic attack, mood fluctuation, manic behavior, depression, manic-depressive behavior, Alzheimer's disease, Huntington's disease, Parkinson's disease, and epilepsy.

23. A method of producing a compound according to claim 15, the method comprising: 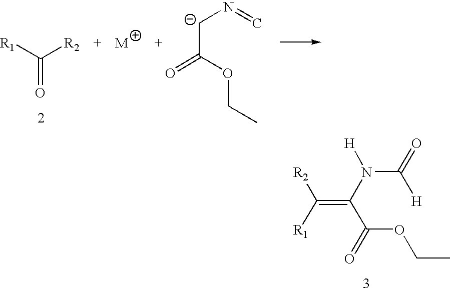
a) deprotonating ethyl isocyanoacetate with a base, and reacting the deprotonating ethyl isocyanoacetate with a ketone of Formula 2, in which R¹ is methyl, and R² is selected from the group consisting of —(CH₂)₆CH₃, —(CH₂)₇CH₃, cyclobutyl, and cyclohexyl, to produce ethyl (E,Z)-2-formylaminoacrylates of Formula 3, 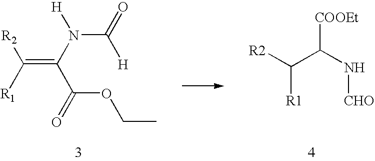
b) reacting ethyl (E,Z)-2-formylaminoacrylate of Formula 3 with Pd/H₂ to produce a formylamino ethyl ester of Formula 4, and 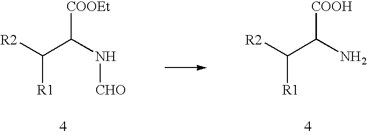
c) reacting the formylamino ethyl ester of Formula 4 with an acid, to produce an amino acid of Formula 1.

24. A method according to claim 23, wherein step (a) is carried out in tetrahydrofuran.

25. A method according to claim 23, wherein in step (c) the acid is hydrochloric acid.

26. A method according to claim 23, further comprising, at a suitable stage, separating the disereomers of the compound, or enatiomers of the compound, by means of HPLC, column chromatography or crystallization.
